(19) United States
(12) Patent Application Publication
(10) Pub. No.: US 2024/0074841 A1
(43) Pub. Date: Mar. 7, 2024

(54) DEVICE AND METHODS FOR PROVIDING ORAL CARE TO INTUBATED SUBJECTS

(71) Applicant: Humberto Nino, Bakersfield, CA (US)

(72) Inventor: Humberto Nino, Bakersfield, CA (US)

(21) Appl. No.: 18/455,619

(22) Filed: Aug. 24, 2023

Related U.S. Application Data
(60) Provisional application No. 63/373,345, filed on Aug. 24, 2022.

(51) Int. Cl. A61C 17/10

(52) U.S. Cl. CPC A61C 17/10; A61M 16/04

(57) ABSTRACT

An oral care device and a method of protecting a subject's tongue with the oral care device is disclosed. The device can include a cup and a stem, the cup having an opening configured to receive a human tongue, and the stem coupled to a closed end of the cup opposite the opening. The cup can include the opening at a first end, a neck portion adjacent to the first end, and a bulb portion between the neck portion and the closed end; and the bulb portion can be configured to receive and retain fluids secreted by the human tongue.

### CROSS-REFERENCE TO RELATED APPLICATIONS

This application claims the benefit of U.S. Provisional Application No. 63/373,345, filed Aug. 24, 2022, the disclosure of which is incorporated herein by reference in its entirety.

### FIELD

The disclosure relates to medical devices, and more particularly to medical devices for providing oral care to intubated subjects.

### BACKGROUND

Tracheal intubation is a process in which a healthcare service provider inserts an endotracheal tube through a patient's mouth and into the trachea to maintain an open airway. After the trachea has been intubated, the tube is left in place to prevent respiratory gases from leaking and to prevent undesirable material such as stomach acid from reaching the tracheobronchial tree. The tube is left in the trachea until ventilatory assistance or protection of the airway is no longer needed. The period of intubation can last weeks. Over the time of intubation, the patient's tongue is often in contact with the tube, which can cause sores and inflammation to the tongue. For example, when fluid secretions around the tongue and mouth floor are not properly drained, the patient's tongue can be become enlarged and forced out of their mouth. Further issues that arise during the use of an endotracheal tube include ulcers, sores, dried and/or cracked tongue, bacterial infections, and tongue necrosis. In some cases, during an epileptic seizure, a patient is at risk of biting their tongue. For at least these reasons, there is a need in the art for a device to protect a patient's tongue during periods of intubation.

### SUMMARY

In various embodiments, an oral care device is provided; the oral device comprising: a cup and a stem, the cup comprising an opening configured to receive a human tongue, and the stem coupled to a closed end of the cup opposite the opening; wherein the cup comprises the opening at a first end, a neck portion adjacent to the first end, and a bulb portion between the neck portion and the closed end; and wherein the bulb portion is configured to receive and retain fluids secreted by the human tongue.

In some embodiments, the cup comprises a hyperboloid three-dimensional shape defined by the first end, neck portion, and bulb portion; and wherein the neck portion comprises an external circumferential arc length that is less than the respective external circumferential arc lengths of the first end and the bulb portion.

In some embodiments, the neck portion comprises a crest defined by a raised interior surface, the crest configured to restrict fluid present in the bulb portion from flowing toward the opening at the first end.

In some embodiments, the bulb portion comprises a well that is configured to receive and retain the fluids secreted by the human tongue.

In some embodiments, the crest is a first spaced distance from a longitudinal axis extending from a central position of the stem, the well is a second spaced distance from the longitudinal axis, and the second spaced distance is greater than the first spaced distance.

In some embodiments, the bulb portion comprises a well configured to receive and retain the fluids secreted by the human tongue, the well having a floor that is about 5 mm to about 20 mm further from a longitudinal axis extending from a central position of the stem relative to the neck portion.

In some embodiments, the cup opening comprises a rim having a discontinuous circular cross-sectional structure that is interrupted by a pair of cut-away portions positioned opposite to one another about the rim.

In some embodiments, the stem comprises an elongated structure having a proximate end coupled to the cup and a distal end that is longitudinally spaced apart from the proximate end.

In some embodiments, the device further comprises a fastener coupled to the distal end of the stem, the fastener configured to attach the oral care device to another device.

In some embodiments, the fastener comprises a pair of straps configured to couple to one another.

In some embodiments, a through-hole is formed while the pair of straps are coupled to one another.

In some embodiments, the well comprises one or more drainage holes.

In various embodiments, a method of protecting a subject's tongue, the subject having an endotracheal tube inserted into the subject's mouth and trachea, the method comprising: providing an oral care device, the oral care device comprising: a cup and a stem, the cup comprising an opening configured to receive a human tongue, and the stem coupled to a closed end of the cup opposite the opening; wherein the cup comprises the opening at a first end, a neck portion adjacent to the first end, and a bulb portion between the neck portion and the closed end; and wherein the bulb portion is configured to receive and retain fluids secreted by the human tongue; and inserting the subject's tongue into the cup.

In some embodiments of the method, the cup comprises a hyperboloid three-dimensional shape defined by the first end, neck portion, and bulb portion; and wherein the neck portion comprises an external circumferential arc length that is less than the respective external circumferential arc lengths of the first end and the bulb portion.

In some embodiments of the method, the neck portion comprises a crest defined by a raised interior surface, the crest configured to restrict fluid present in the bulb portion from flowing toward the opening at the first end.

In some embodiments of the method, the bulb portion comprises a well that is configured to receive and retain the fluids secreted by the human tongue.

In some embodiments of the method, the crest is a first spaced distance from a longitudinal axis extending from a central position of the stem, the well is a second spaced distance from the longitudinal axis, and the second spaced distance is greater than the first spaced distance.

In some embodiments of the method, the stem comprises an elongated structure having a proximate end coupled to the cup and a distal end that is longitudinally spaced apart from the proximate end.

In some embodiments of the method, the oral care device further comprises a fastener coupled to the distal end of the stem, the fastener configured to attach the oral care device to an object in proximity to the subject.

In some embodiments, the method further comprises a step of coupling the fastener to the object in proximity to the subject.

Additional features and advantages of the embodiments disclosed herein will be set forth in the detailed description that follows, and in part will be clear to those skilled in the art from that description or recognized by practicing the embodiments described herein, including the detailed description which follows, the claims, as well as the appended drawings.

Both the foregoing general description and the following detailed description present embodiments intended to provide an overview or framework for understanding the nature and character of the embodiments disclosed herein. The accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS

The features and advantages of the present disclosure will be more fully described in, or rendered obvious by the following detailed description of the preferred embodiments, which are to be considered together with the accompanying drawings wherein like numbers refer to like parts and further, wherein:

FIG. 1 is a right-side view of an oral care device, in accordance with some embodiments described herein;

FIG. 2 is a left-side view of the oral care device of FIG. 1;

FIG. 3A is a front view of the oral care device of FIG. 1, in accordance with some embodiments described herein;

FIG. 3B is a front view of the oral care device of FIG. 1, in accordance with some embodiments described herein;

FIG. 4 is top view of the oral care device of FIG. 1;

FIG. 5 is a cross-sectional side view taken about the 5-5′ line in FIG. 4;

FIG. 6 is a bottom view of the oral care device of FIG. 1, in accordance with some embodiments described herein;

FIG. 7A is a bottom view of the oral care device of FIG. 1, in accordance with some embodiments described herein;

FIG. 7B is a plug cap configured for the oral care device of FIG. 7A;

FIG. 8 is an illustration showing a human subject with an endotracheal tube inserted into their mouth and trachea, and the oral care device of FIG. 1 prior to engaging with the subject's tongue, in accordance with some embodiments described herein; and

FIG. 9 is an illustration showing the oral anatomy of a human subject and the oral care device of FIG. 1 engaged with the subject's tongue, in accordance with some embodiments described herein.

The drawings are not necessarily to scale, and certain features and certain views of the drawings may be shown exaggerated in scale or in schematic in the interest of clarity and conciseness.

### DETAILED DESCRIPTION

Reference will now be made in detail to the present preferred embodiment(s), and examples of which is/are illustrated in the accompanying drawings. Whenever possible, the same reference numerals will be used throughout the drawings to refer to the same or like parts. Any specific details of the embodiments are used for demonstration purposes only, and no unnecessary limitations or inferences are to be understood therefrom.

Before describing the exemplary embodiments, it is noted the embodiments reside primarily in combinations of components and procedures related to the device. Accordingly, the device components and method procedures have been represented where appropriate in the drawings, showing only those specific details that are pertinent to understanding the embodiments of the present disclosure so as not to obscure the disclosure with details that will be readily apparent to those of ordinary skill in the art having the benefit of the description herein.

The specific details of the various embodiments described herein are used for demonstration purposes only, and no unnecessary limitation or inferences are to be understood therefrom. For example, references to “various embodiments,” “some embodiments,” “other embodiments,” “an example embodiment,” etc., indicate that the embodiment described may include a particular feature, structure, or characteristic, but every embodiment may not necessarily include that feature, structure, or characteristic. Such phrases are not necessarily referring to the same embodiment. When a particular feature, structure, or characteristic is described in connection with an embodiment, it is within the knowledge of one skilled in the art to affect such feature, structure, or characteristic in connection with other embodiments whether explicitly described or not.

The specific details of the various embodiments described herein are used for demonstration purposes only, and no unnecessary limitation or inferences are to be understood therefrom. Furthermore, as used herein, relational terms, such as “first” and “second,” “top” and “bottom,” and the like, may be used solely to distinguish one entity or element from another entity or element without necessarily requiring or implying any physical or logical relationship, or order between such entities or elements.

As used herein, the term “intubate” or “intubated” refers to the insertion of a tube into a hollow organ or body passage. As used herein, the term “endotracheal intubation” refers to the passage of a tube through the nose or mouth into the trachea for maintenance of the airway.

As used herein, the terms “subject,” “individual,” or “patient” can be an individual organism, a vertebrate, a mammal, or a human. “Mammal” includes a human, non-human primate, murine (e.g., mouse, rat, guinea pig, hamster), ovine, bovine, ruminant, lagomorph, porcine, caprine, equine, canine, feline, avis, etc.

In various embodiments, an oral care device and a method of using the oral care device to treat a subject are provided. In some embodiments, the oral care device comprises a component configured to receive the subject's tongue, a component for allowing a user to grasp the oral care device, and a component for coupling the oral care device to another device. In various embodiments, a method of treating a subject is provided. In some embodiments, the method comprises removably attaching the oral care device to the tongue of the subject, and removably attaching the oral care device to the other device.

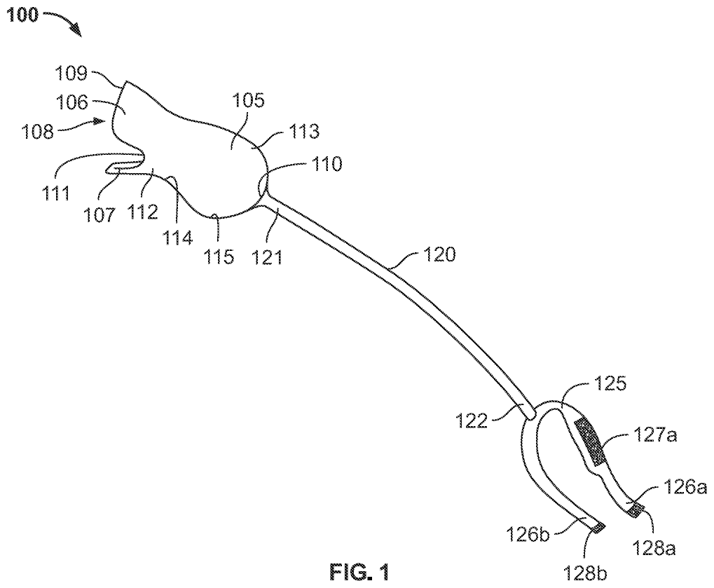

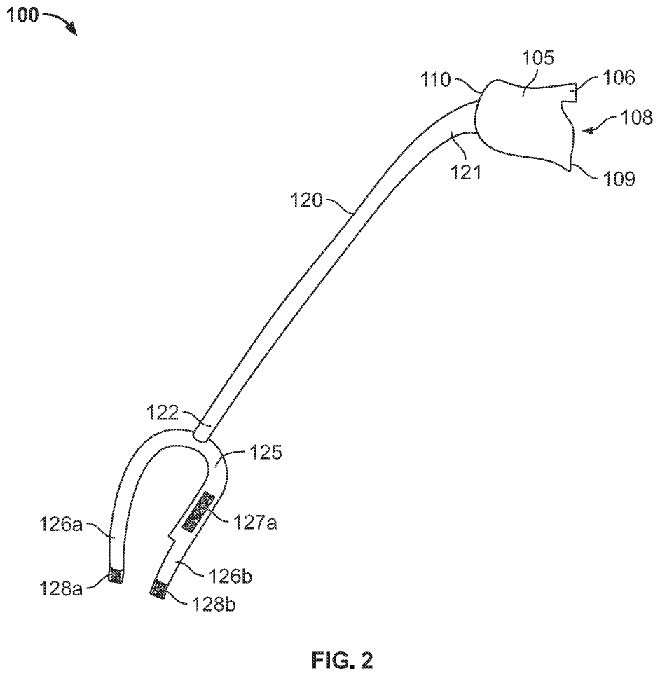

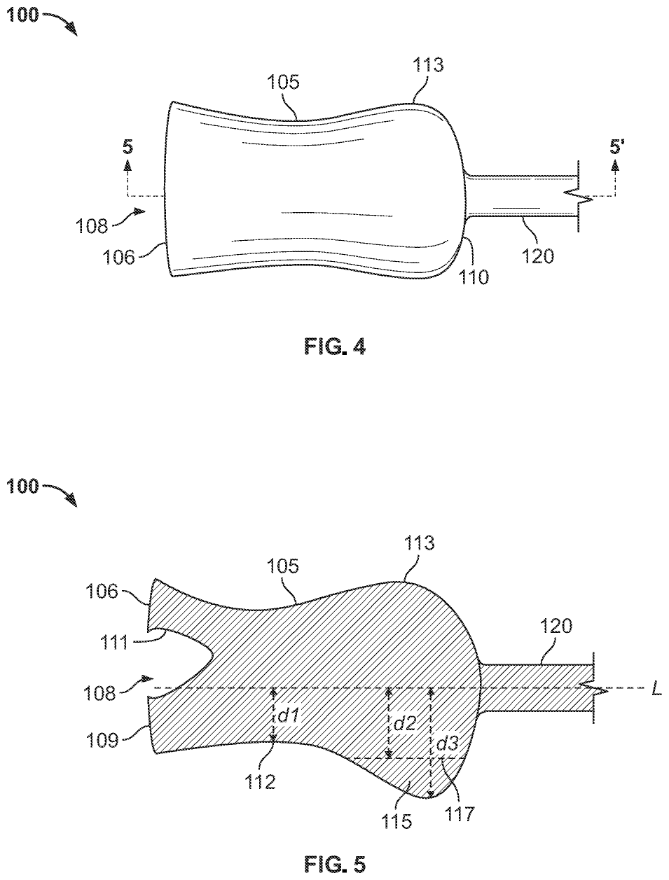

In various embodiments, as shown in FIGS. 1-9, the oral care device 100 is provided. In some embodiments, as shown in FIGS. 1 and 2, the oral care device 100 comprises the cup 105, the stem 120, and the fastener 125. In such embodiments, the cup 105 is sized, shaped, and generally configured to receive the tongue of a subject. In some embodiments, the subject is a human and the cup 105 is sized and shaped to receive the distal portion of the human tongue. In some embodiments, the cup 105 comprises smooth internal and external surfaces. In some embodiments, the cup 105 does not include flanges or other components projecting from the external surface. In some embodiments, the cup 105 protects the tongue from damage, including for example, sores caused from the tongue rubbing against an endotracheal tube.

In some embodiments, the cup 105 comprises a generally cylindrical (e.g., hyperboloid or hourglass) three-dimensional (3D) shape having the open first end 109 and the closed second end 110. In some embodiments, the first end 109 comprises a rim that forms the opening 108 through which a tongue can be inserted into. In some embodiments, the rim comprises a generally circular or partially circular (e.g., an incomplete circle with two complementary semicircles) cross-sectional shape. In some embodiments, the second end 110 is a closed structure having a wall surface that does not allow the tongue to pass through. In some embodiments, the rim formed at the first end 109 comprises a circumferential arc length that is sufficiently large enough to receive the distal portion of a human tongue. In some embodiments, the first end 109 is sized and shaped to fit inside of a human mouth.

In some embodiments, the first end 109 comprises a rim that is discontinuous about the circumferential perimeter (e.g., two complementary semicircles with their respective concave portions facing one another). In such embodiments, the first end 109 comprises one or more cut-away portions 111 (e.g., two cut-away portions positioned opposite of one another on opposing left and right sides of the cup 105) that form an ergonomic structure for receiving the human tongue and the tissue between the tongue and mouth floor. In such embodiments, the cut-away portion 111 divides the cup 105 into the upper portion 106 and the lower portion 107. In some embodiments, the upper portion 106 is configured to be inserted into a subject's mouth between the tongue and the roof of the mouth, and the lower portion 107 is configured to be inserted between the tongue and the floor of the mouth.

In some embodiments, the first end 109 comprises a rim that is continuous about the circumferential perimeter. In such embodiments, the one or more cut-away portions 111 are absent, allowing for a generally circular rim about the first end 109.

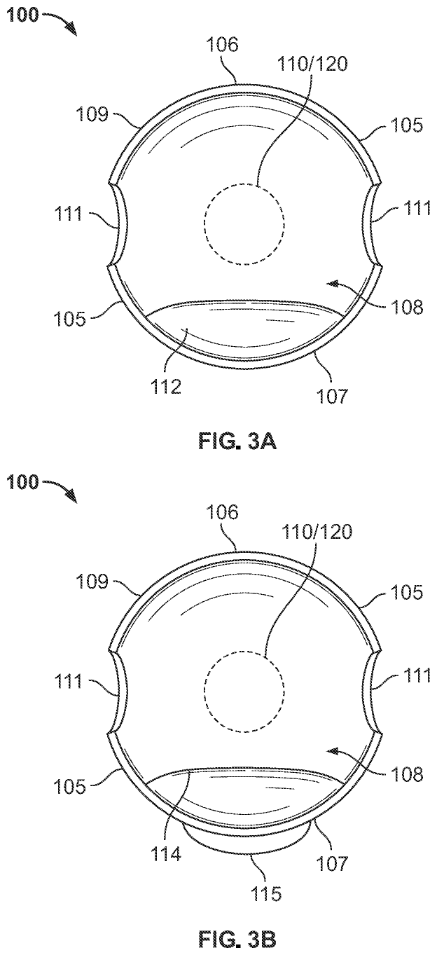

In some embodiments, as shown in FIG. 3A-FIG. 5, the cup 105 comprises a hyperboloid or hourglass three-dimensional shape defined by the opening 108 at the first end 109, the neck portion 112, the bulb portion 113, and the second end 110. In such embodiments, the neck portion 112 comprises an external circumferential arc length that is less than the respective external circumferential arc lengths of the first end 109 and the bulb portion 113. In such embodiments, the relatively smaller circumferential arc length about the neck portion 112 allows for the crest 114 to be formed in the interior surface of the cup 105. In some embodiments, as shown in FIG. 5, the crest 114 is the position on the interior surface of the cup 105 that is closest in distance to the longitudinal axis L defined by the stem 120 when the stem is coupled to the center position on the second end 110.

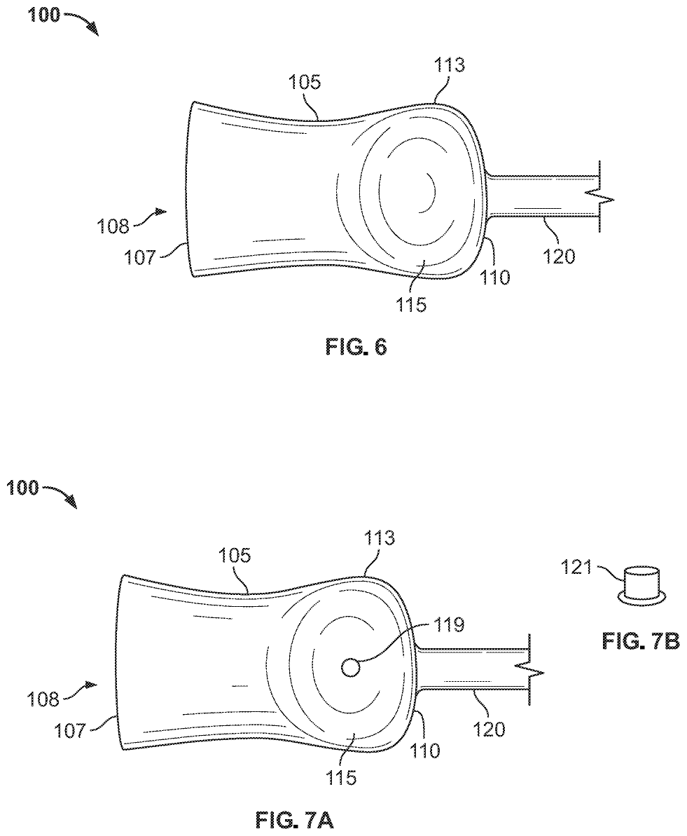

In some embodiments, as shown in FIGS. 5 and 6, the bulb portion 113 comprises the well 115, which is a depression positioned between the crest 114 and the second end 110. In such embodiments, the well 115 is configured to receive and contain fluids present inside the cup 105. In some embodiments, as shown in FIG. 5, the well 115 is the position on the interior surface of the cup 105 that is furthest in distance from the longitudinal axis L. In such embodiments, any fluids contained inside the cup 105 will settle in the well 115 and be unable to pass over the crest 114 toward the first end 109. In some embodiments, the crest 114 is configured to prevent or reduce the chances of fluids contained in the well 115 from re-entering the subject's mouth during use.

In some embodiments, the distance between the well 115 and the longitudinal axis L will determine the amount of fluid that can be contained and retained during use. The amount of fluid secreted by the subject's tongue and/or mouth will depend on the individual and their condition. In some embodiments, the subject may not produce much fluid and the floor of the well 115 can be relatively shallow. As shown in FIG. 5, the relative depth of the well 105 is indicated by the line 117, which is a distance d2 from the longitudinal axis L. The distance d2 is greater than the distance d1, which spans between the crest 112 and the longitudinal axis L. In some embodiments, the subject may produce a larger amount of fluid. In such embodiments, the cup 105 comprises a well 115 with a substantially deeper floor than the well line 117 or the crest 114. As shown in FIG. 5, for example, the well 115 is a distance d3 from the longitudinal axis L. The distance d3 is substantially greater than the distance d1, which spans between the crest 112 and the longitudinal axis L.

In some embodiments, the distance d3 is about 10% to about 75% greater than the distance d1. In some embodiments, the distance d3 is about 15%, about 20%, about 25%, about 30%, about 35%, about 40%, about 45%, about 50%, or about 55% greater than the distance d1. In some embodiments, the distance d3 and the distance d1 are defined by a ratio ranging from about 1.1:1 to about 5:1, including about 1.5:1; about 2:1; about 2.5:1; or about 3:1.

In some embodiments, the floor of the well 115 is deeper than the crest 114 by a predetermined distance ranging from about 5 mm to about 50 mm, including any subranges therebetween (e.g., about 10 mm to about 30 mm, about 15 mm to about 25 mm, etc.) and any intermediate points (e.g., about 10 mm, 15 mm, 17.5 mm, 20 mm, 30 mm, etc.). In some embodiments, the deeper the floor of the well 115 sits relative to the crest 114, the more liquid (e.g., saliva or oral secretions) can be retained during use.

In some embodiments, the well 115 comprises one or more drainage holes configured to release any fluids contained in the cup 105. As shown in FIG. 7A, for example, the well 115 comprises the drainage hole 119. The drainage hole 119 is positioned on the well 115 where it is capable of draining the fluid contained in the well 115. In some embodiments, as shown in FIG. 7B, the oral care device 100 comprises a plug cap 121 configured to close the drainage hole 119. Any suitable combination of drainage hole and plug cap can be used. For example, the drainage hole 119 can be a through-hole and the plug cap can be a tapered cylindrical structure that can be removably inserted into the through-hole. In some embodiments, the plug cap includes an insertion body and a corresponding arm, whereby the insertion body can be inserted into the drainage hole 119 and the arm can be used to couple the plug cap 121 to the insertion body. In such embodiments, when the plug cap 121 is pulled out of the drainage hole 119, it will remain attached to the well 115 via the insertion body.

In some embodiments, the oral care device 100 comprises the stem 120. In some embodiments, the stem 120 comprises a longitudinal tube or rod structure extending between a first end 121 and a second end 122. In some embodiments, the first end 121 of the stem 120 is coupled to the second end 110 of the cup 105. In some embodiments, the first end 121 of the stem 120 is permanently coupled to the second end 110 of the cup 105. In some embodiments, the first end 121 of the stem 120 is removably coupled to the second end 110 of the cup 105. In such embodiments, the stem 120 and cup 105 can be coupled using any suitable means, wherein the suitability includes maintaining a clean and/or sterile environment in a hospital setting. In some embodiments, for example, the stem 120 and cup 105 can be coupled using one or more hook-and-loop fasteners (e.g., Velcro), buckles, snaps, hook and eye closures, magnets, etc.

In some embodiments, the second end 122 of the stem 120 is coupled to the fastener 125. The stem 120 can be any suitable length. In this context, the suitability of the length refers to the ability of a user to grasp and control the oral care device 100 for the purpose of inserting a subject's tongue into the cup 105, and further refers to the ability to connect the fastener 125 to another, separate device within proximity to the subject (e.g., near the subject's mouth, chin, throat, shoulder, or chest). In some embodiments, for example, the stem 120 has a length ranging from about 2 inches to about 14 inches, including any subranges any intermediate points therebetween.

In some embodiments, the oral care device 100 comprises the fastener 125. In some embodiments, the fastener 125 is connected to the second end 122 of the stem 120. In such embodiments, the fastener 125 is configured to removably attach the second end 122 to a separate object, for example, to another device or structure in proximity to the oral care device 100. In some embodiments, the fastener 125 comprises the straps 126a and 126b. In some embodiments, the straps 126a, 126b are configured to be coupled to one another. For example, in some embodiments, the straps 126a, 126b comprise an adequate length for independently wrapping around another object and then to one another for the purpose of securing the oral care device 100 to the object. In some embodiments, each of the straps 126a, 126b respectively comprise one or more complementary coupling mechanisms. In some embodiments, for example, the complementary coupling mechanisms comprises one or more hook-and-loop fasteners (e.g., Velcro), buckles, snaps, hook and eye closures, magnets, etc. In some embodiments, the straps 126a, 126b are, independently from one another, length-adjustable and can each be adjusted to a longer or shorter length.

The oral care device, or components thereof, that are described herein can be comprised of any suitable material. In some embodiments, for example, the oral care device or individual components thereof are comprised of one or more plastic compositions (e.g., thermoplastics, including polyurethane). In some embodiments, for example, the oral care device, or individual components thereof, is comprised of one or more milled or cast metal or metal alloys. In some embodiments, the oral care device or individual components thereof are comprised of a medical grade material. In some embodiments, the oral care device or individual components thereof are comprised of a medical grade material that can be sterilized with existing techniques (e.g., autoclave, ethylene oxide). In some embodiments, the oral care device is comprised of a single, unitary piece of material. In other embodiments, the oral care device is comprised of a plurality of components that are assembled (e.g., coupled together) to form a unitary device.

In various embodiments, the external surface of the oral care device 100 does not include flanges or other components projecting outward from the surface thereof. In such embodiments, a caregiver can access the subject's mouth without removing the oral care device from the subject's tongue. In such embodiments, the caregiver would be able to, for example, insert a thermometer into the subject's mouth, brush the subject's teeth with a toothbrush, suction fluid from the oral cavity with a suction catheter, etc., while the oral care device 100 is engaged with the subject's tongue.

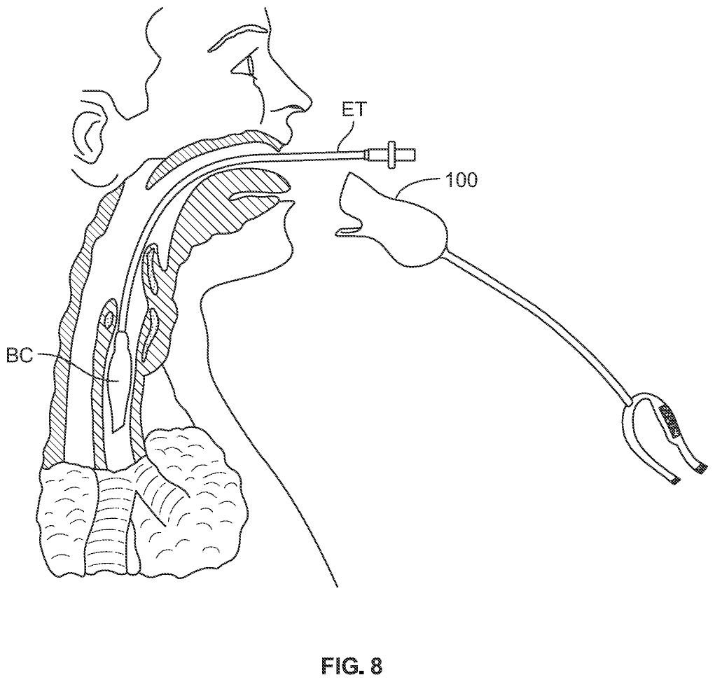

In various embodiments, the oral care device described herein is configured for use in a medical environment, including, for example, a hospital setting. As would be appreciated by one skilled in the art, certain hospital patients require oral intubation, including endotracheal intubation. As shown in FIG. 8, endotracheal intubation involves the insertion of a flexible plastic endotracheal tube (ET) into the subject's mouth and trachea to maintain an open airway. The internal, distal end of the endotracheal tube (ET) can include the balloon cuff (BC), which can be inflated to secure the tube in place, prevent the leakage of respiratory gases, and protect the tracheobronchial tree from receiving undesirable materials such as stomach acid. The opposite, external end of the endotracheal tube (ET) can be coupled to an instrument, such as another tube, pump, or ventilator. FIG. 8 shows the endotracheal tube (ET) inserted into the subject's mouth and the oral care device 100 prior to its use with the subject.

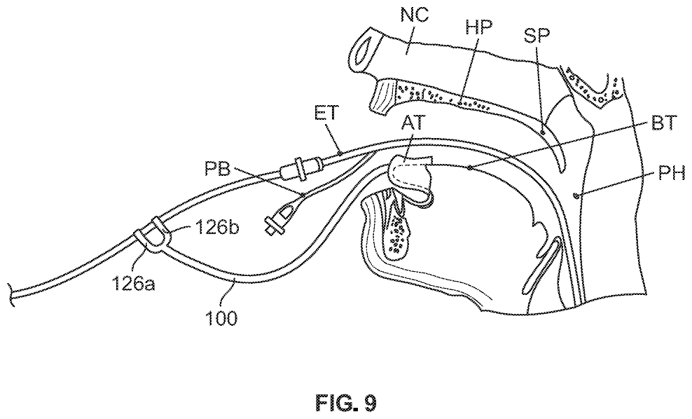

FIG. 9 shows the oral anatomy of a subject, the endotracheal tube (ET) inserted into a subject's mouth and pharynx (PH), and the oral care device 100 attached to the apex of the subject's tongue (AT). The oral anatomy includes the nasal cavity (NC), the hard palate (HP), the soft palate (SP), and the pharynx (PH). The endotracheal tube (ET) comprises a pilot balloon (PB) for controlling the balloon cuff (BC) at the internal, distal end. As shown in FIG. 9, the endotracheal tube (ET) is inserted into the subject's mouth and extends over the body of the tongue (BT) through the pharynx (PH) toward the trachea. As would be appreciated by one skilled in the art, the external end of endotracheal tube (ET) comprises a plug for connecting the tube to another tube or an instrument, such as a pump or ventilator. In such situations, the endotracheal tube (ET) extends outward from the subject's mouth and can function as a support for the oral care device 100 to couple to. In such embodiments, the straps 126a, 126b can be coupled to the endotracheal tube (ET) or to a separate device (e.g., tube) that is removably coupled to the plug of the endotracheal tube (ET).

In various embodiments, as shown in FIGS. 8 and 9, a method of using the oral care device 100 is provided. FIG. 8 shows the endotracheal tube (ET) inserted into the subject's mouth and trachea to maintain an open airway. In some embodiments, the method comprises the step of inserting the endotracheal tube into the subject's mouth and trachea. FIG. 9 shows the oral care device 100 engaged with the apex of the subject's tongue (AT) (i.e., the tongue is inserted into the oral care device 100). In some embodiments, the method comprises the step of inserting the distal portion of the subject's tongue into the cup 105 of the oral care device 100. In some embodiments, the method comprises attaching the pair of straps 126a, 126b around the endotracheal tube (ET) or a separate device (e.g., tube) that is removably coupled to the endotracheal tube (ET). In some embodiments, the method comprises inserting a thermometer, brush, suction catheter, or other device (e.g., toothbrush) into the subject's mouth while the oral care device 100 is concurrently attached to the subject's tongue.

The oral care device 100 and the methods of using the oral care device to treat a subject provide several unexpected benefits. Subjects having an endotracheal tube inserted into their mouth are susceptible to various undesirable side effects caused by the endotracheal tube. For example, when fluid secretions around the tongue and mouth floor are not properly drained, the subject's tongue can be become enlarged and forced out of their mouth. Further issues that arise during the use, including the prolonged use, of an endotracheal tube include ulcers, sores, dried and/or cracked tongue, bacterial infections, and tongue necrosis. In some cases, during an epileptic seizure, a subject is at risk of biting their tongue, and the oral care device 100 would prevent the subject's teeth from contacting the tongue in a biting motion. The method of using the oral care device 100 disclosed herein allows for improved patient hygiene and comfort while reducing the risk of undesired counterindications due to the use of an endotracheal tube.

Exemplary embodiments of the device and methods are described above in detail. The device and methods are not limited to the specific embodiments described herein, but rather, components of the device and/or steps of the method may be utilized independently and separately from other components and/or steps described herein. For example, the device may also be used in combination with other methods and is not limited to practice with a method as described herein. Although specific features of some embodiments may be shown in some drawings and not in others, this is for convenience only. In accordance with the principles of the disclosure, any feature of a drawing may be referenced and/or claimed in combination with any feature of any other drawing.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, the use of examples, or exemplary language (e.g., “such as”), is intended to illuminate the embodiments and does not pose a limitation on the scope of the claims unless otherwise stated. No language in the specification should be construed as indicating any non-claimed element as essential.

As used herein, the terms “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

This written description uses examples to disclose various embodiments, including the best mode, and to enable those skilled in the art to practice the embodiments, including making and using any devices and performing any methods. The patentable scope of the present embodiments is defined by the claims, and may include other examples that occur to those skilled in the art, which are intended to be within the scope of the claims if they have structural elements that do not differ from the literal language of the claims, or if they include equivalent structural elements with insubstantial differences from the language of the claims.

I/We claim:

1. An oral care device, comprising:
a cup and a stem, the cup comprising an opening configured to receive a human tongue, and the stem coupled to a closed end of the cup opposite the opening;
wherein the cup comprises the opening at a first end, a neck portion adjacent to the first end, and a bulb portion between the neck portion and the closed end; and
wherein the bulb portion is configured to receive and retain fluids secreted by the human tongue.

2. The oral care device of claim 1, wherein the cup comprises a hyperboloid three-dimensional shape defined by the first end, neck portion, and bulb portion; and
wherein the neck portion comprises an external circumferential arc length that is less than the respective external circumferential arc lengths of the first end and the bulb portion.

3. The oral care device of claim 1, wherein the neck portion comprises a crest defined by a raised interior surface, the crest configured to restrict fluid present in the bulb portion from flowing toward the opening at the first end.

4. The oral care device of claim 3, wherein the bulb portion comprises a well that is configured to receive and retain the fluids secreted by the human tongue.

5. The oral care device of claim 4, wherein the crest is a first spaced distance from a longitudinal axis extending from a central position of the stem, the well is a second spaced distance from the longitudinal axis, and the second spaced distance is greater than the first spaced distance.

6. The oral care device of claim 1, wherein the bulb portion comprises a well configured to receive and retain the fluids secreted by the human tongue, the well having a floor that is about 5 mm to about 20 mm further from a longitudinal axis extending from a central position of the stem relative to the neck portion.

7. The oral care device of claim 1, wherein the cup opening comprises a rim having a discontinuous circular cross-sectional structure that is interrupted by a pair of cut-away portions positioned opposite to one another about the rim.

8. The oral care device of claim 1, wherein the stem comprises an elongated structure having a proximate end coupled to the cup and a distal end that is longitudinally spaced apart from the proximate end.

9. The oral care device of claim 8, further comprising a fastener coupled to the distal end of the stem, the fastener configured to attach the oral care device to another device.

10. The oral care device of claim 9, wherein the fastener comprises a pair of straps configured to couple to one another.

11. The oral care device of claim 10, wherein a through-hole is formed while the pair of straps are coupled to one another.

12. The oral care device of claim 1, wherein the well comprises one or more drainage holes.

13. A method of protecting a subject's tongue, the subject having an endotracheal tube inserted into the subject's mouth and trachea, the method comprising:
providing an oral care device, the oral care device comprising:
a cup and a stem, the cup comprising an opening configured to receive a human tongue, and the stem coupled to a closed end of the cup opposite the opening;
wherein the cup comprises the opening at a first end, a neck portion adjacent to the first end, and a bulb portion between the neck portion and the closed end; and
wherein the bulb portion is configured to receive and retain fluids secreted by the human tongue; and
inserting the subject's tongue into the cup.

14. The method of claim 13, wherein the cup comprises a hyperboloid three-dimensional shape defined by the first end, neck portion, and bulb portion; and
wherein the neck portion comprises an external circumferential arc length that is less than the respective external circumferential arc lengths of the first end and the bulb portion

15. The method of claim 13, wherein the neck portion comprises a crest defined by a raised interior surface, the crest configured to restrict fluid present in the bulb portion from flowing toward the opening at the first end.

16. The method of claim 15, wherein the bulb portion comprises a well that is configured to receive and retain the fluids secreted by the human tongue.

17. The method of claim 16, wherein the crest is a first spaced distance from a longitudinal axis extending from a central position of the stem, the well is a second spaced distance from the longitudinal axis, and the second spaced distance is greater than the first spaced distance.

18. The method of claim 13, wherein the stem comprises an elongated structure having a proximate end coupled to the cup and a distal end that is longitudinally spaced apart from the proximate end.

19. The method of claim 18, wherein the oral care device further comprises a fastener coupled to the distal end of the stem, the fastener configured to attach the oral care device to an object in proximity to the subject.

20. The method of claim 19, further comprising coupling the fastener to the object in proximity to the subject.
